(19) United States
(12) Patent Application Publication
(10) Pub. No.: US 2015/0152080 A1
(43) Pub. Date: Jun. 4, 2015

(54) BENZOTHIOPHENE SULFONAMIDES DERIVATIVES AS CHEMOKINE RECEPTOR MODULATORS

(71) Applicant: Allergan, Inc., Irvine, CA (US)

(72) Inventors: Haiqing Yuan, Irvine, CA (US); Xiaoxia Liu, Lake Forest, CA (US); Richard L. Beard, Newport Beach, CA (US)

(21) Appl. No.: 14/557,912

(22) Filed: Dec. 2, 2014

Related U.S. Application Data
(60) Provisional application No. 61/910,494, filed on Dec. 2, 2013.

(51) Int. Cl. C07D 333/62; C07D 409/12

(52) U.S. Cl. CPC C07D 333/62; C07D 409/12

(57) ABSTRACT

The present invention relates to benzothiophene sulfonamide derivatives, processes for preparing them, pharmaceutical compositions containing them and their use as pharmaceuticals as modulators of chemokine receptors.

### CROSS-REFERENCE TO RELATED APPLICATIONS

This application claims the benefit of U.S. provisional application 61/910,494 entitled “Benzothiophene Sulfonamides Derivatives As Chemokine Receptor Modulators” filed on Dec. 2, 2013, which is incorporated herein by reference in its entirety and serves as the basis of a priority claim.

### FIELD OF THE INVENTION

The present invention relates to novel benzothiophene sulfonamide derivatives, processes for preparing them, pharmaceutical compositions containing them and their use as pharmaceuticals as modulators of chemokine receptors. The invention relates specifically to the use of these compounds and their pharmaceutical compositions to treat disorders associated with chemokine receptor modulation.

### BACKGROUND OF THE INVENTION

Chemokines are a group of 7- to 14-kd peptides that play an important role in orchestrating leukocyte recruitment and migration during inflammation, and therefore represent an important target for anti-inflammatory therapies (Wells et al., 2006). They act by binding to seven-transmembrane, G protein-coupled receptors, the chemokine receptors. The chemokine system is complex, with about 50 chemokines and 20 chemokine receptors identified in humans, often acting with redundancy, making selection of specific antagonists difficult (Gerard and Rollins, 2001). Genetic knockout strategies have confirmed the importance of chemokines as regulators of immune function, but the deletion of specific chemokines has led to only specific and relatively mild defects in the inflammatory response further emphasizing the complex redundancy of the system. Selectivity is crucial for use of chemokine receptor antagonists in systemic diseases where a single chemokine-receptor system is implicated such as atheroscelorsis where the macrophage/monocyte system is the major player in order to allow a subtle and specific control over immune function (Weisberg et al., 2006; Feria and Diaz Gonzalez et al., 2006).

Many ocular conditions are characterized by inappropriate migration and infiltration of cells such as leukocytes and endothelial cells into the eye with deleterious effects to ocular structures (Wallace et al., 2004). Chemokines have been identified in such diseases and misregulation of the chemokine system is apparent in corneal graft rejection, diabetic retinopathy, age-related macular degeneration (ARMD), chronic inflammatory diseases such as uveitis, dry eye etc. Mice lacking CCR2 or MCP-1 develop features of ARMD with age, including drusen deposits, choroidal neovascularization and photoreceptor atrophy indicating a crucial role for this chemokine and its receptor signaling (Amabati et al., 2003). Thus CCR2 receptor-specific inhibitor might have potential therapeutic benefit in ocular diseases like ARMD. In contrast, various human and animal studies have identified several chemokines in different forms of uveitis, produced both by resident and infiltrating cells, that strongly suggests a prominent role for these molecules in its pathogenesis. Studies in rat and mice models of uveitis have demonstrated up-regulation of monocyte chemoattractant protein-1 (MCP-1), macrophage inflammatory protein-1 (MIP-1), RANTES, stromal derived factor-1 (SDF-1) which are powerful chemoattractants for monocytes and T-cells (Fang et al., 2004; Keino et al., 2003). Similar findings have been reported in peripheral blood mononuclear cells in patients with acute anterior uveitis (AAU), the most common form of human uveitis (Klitgaard et al., 2004). MCP-1 knockout mice and CCR5 knockout mice show reduced endotoxin-induced uveitis, which is the animal model for AAU (Takeuchi et al., 2005; Tuallion et al., 2002). It has also been demonstrated that blocking the chemokine system upstream with the use of NF-κB blockers significantly attenuates experimental AAU in rats (Yang et al., 2005). Blockage of NF-κB results in transcriptional inhibition of multiple chemokines. Given the complexity of pathogenesis in uveitis it is unlikely that a selective inhibition of a chemokine receptor in monotherapy will offer therapeutic benefit. A similar role of multiple chemokines have been shown to be correlated with clinical stage of disease in diabetic retinopathy and dry eye (Meleth et al., 2005; Yamagami et al., 2005). In these ocular diseases the use of broad spectrum chemokine receptor inhibitor which inhibits the function of a wide range of chemokines may be beneficial.

The first broad spectrum chemokine inhibitor (BSCI) to be reported was termed Peptide 3, which was derived from the sequence of human chemokine MCP-1 and was shown to block the migration of monocytes in response to MCP-1, MIP-1, RANTES and SDF-1 (Reckless and Grainger. 1999). A cyclic retro inverse analogue of Peptide 3, constructed of D-amino acids in the reverse sequence, called NR58-3.14.3 was observed to be a more potent chemokine inhibitor (Beech et al., 2001). NR58-3.14.3 has been used to test for anti-inflammatory activities in animal models of atherosclerosis, lung inflammation, irritable bowel syndrome etc (Beech et al., 2001; Grainger and Reckless. 2003; Tokuyama et al., 2005). However there are several disadvantages to using these BSCI as a long-term therapeutic strategy. The known BSCIs which are peptides which have relatively low potency, poor pharmacokinetics, and are unstable in vivo. In addition, systemic use of broad spectrum chemokine receptor inhibitors could potentially lead to deleterious side effects due to their systemic anti-inflammatory activity. However in ocular diseases, a local or topical application would prevent the broad spectrum inhibitor to be taken up systemically. Identification of a small molecule inhibitor of several chemokine receptors could be very useful for treatment of inflammatory ocular diseases. Given the evidence for the role of multiple chemokines in several ocular diseases and these results, we propose that the use of small and large molecule broad spectrum chemokine receptor inhibitors will have utility in the local treatment of ocular inflammatory diseases including, but not limited to, uveitis, dry eye, diabetic retinopathy, allergic eye disease and proliferative retinopathies.

Manipulation of multiple chemokines therefore represents a novel therapeutic approach in treating ocular diseases.

WO2008008374 discloses CCR2 inhibitors and methods of use thereof.

JP 2003335670 A discloses benzothiophen sulfonamide analogs as bioadhesion inhibitors.

JP 2003267870 A discloses pharmaceuticals containing benzothiophenesulfonamides for prophylactic and therapeutic treatment of pulmonary hypertension.

WO2002022595 A1 discloses the preparation of N-phenylbenzothiophene-sulfonamide derivatives as selective chymase inhibitors.

US2007037794 A1 discloses CCR2 inhibitors and methods of use thereof.

WO2009009740 A1 discloses fused heteroaryl pyridyl and phenyl benzenesulfonamides as CCR2 modulators for the treatment of inflammation.

### SUMMARY OF THE INVENTION

A group of novel benzothiophene sulfonamide derivatives which are potent and selective chemokine receptor modulators, has been now discovered. As such, the compounds described herein are useful in treating a wide variety of disorders associated with modulation of chemokine receptors. The term “modulator” as used herein, includes but is not limited to: receptor agonist, antagonist, inverse agonist, inverse antagonist, partial agonist, partial antagonist.

This invention describes compounds of Formula I, which have chemokine receptor biological activity. The compounds in accordance with the present invention are thus of use in medicine, for example in the treatment of humans with diseases and conditions that are alleviated by chemokine receptor modulation.

In one aspect, the invention provides a compound having Formula I or a pharmaceutically acceptable salt thereof or stereoisomeric forms thereof, or the, enantiomers, diastereoisomers, tautomers, zwitterions and pharmaceutically acceptable salts thereof:

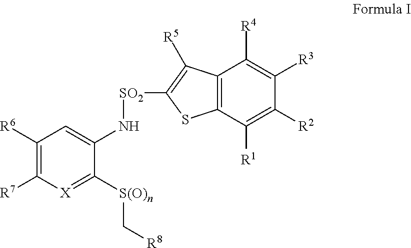

wherein:
X is N or CR;
R is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R¹ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R² is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R³ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁴ is hydrogen, halogen, CN, substituted or unsubstituted C₁₋₆ alkyl, substituted or unsubstituted Cₘ cycloalkyl, OR⁹, NR¹⁰R¹¹ or COR¹²;
R⁵ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁶ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁷ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁸ is substituted or unsubstituted C₁₋₆ alkyl, substituted or unsubstituted C₃₋₈ cycloalkyl, substituted or unsubstituted aryl or substituted or unsubstituted heteroaryl;
n is 0, 1 or 2;
R⁹ is hydrogen or substituted or unsubstituted C₁₋₆ alkyl;
R¹⁰ is hydrogen or substituted or unsubstituted C₁₋₆ alkyl;
R¹¹ is hydrogen, substituted or unsubstituted C₁₋₆ alkyl, substituted or unsubstituted heterocycle or substituted or unsubstituted C₆₋₁₀ aryl;
R¹² is hydrogen, hydroxyl, substituted or unsubstituted heterocycle, substituted or unsubstituted C₆₋₁₀ aryl or substituted or unsubstituted C₁₋₆ alkyl;
except compounds:

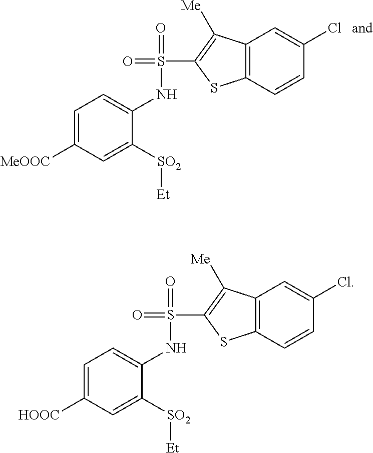 [SMILES: CCS(=O)(=O)c1cc(C(=O)OC)ccc1NS(=O)(=O)c1sc2ccc(Cl)cc2c1C ; CCS(=O)(=O)c1cc(C(=O)O)ccc1NS(=O)(=O)c1sc2ccc(Cl)cc2c1C]

In another aspect, the invention provides a method of treating a disorder associated with chemokine receptor modulation, which comprises administering to a mammal in need thereof, a pharmaceutical composition comprising a therapeutically effective amount of at least one compound of Formula I:

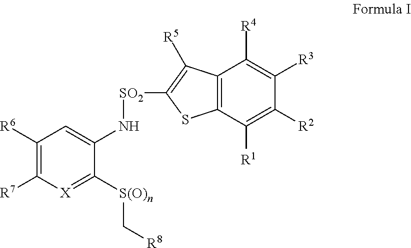

or a pharmaceutically acceptable salt thereof, wherein:
X is N or CR;
R is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R¹ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R² is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R³ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁴ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁵ is hydrogen, halogen, CN, substituted or unsubstituted C₁₋₆ alkyl, substituted or unsubstituted Cₘ cycloalkyl, OR⁹, NR¹⁰R¹¹ or COR¹²;
R⁶ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁷ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁸ is substituted or unsubstituted C₁₋₆ alkyl, substituted or unsubstituted C₃₋₈ cycloalkyl, substituted or unsubstituted aryl or substituted or unsubstituted heteroaryl;
n is 0, 1 or 2;
R⁹ is hydrogen or substituted or unsubstituted C₁₋₆ alkyl;
R¹⁰ is hydrogen or substituted or unsubstituted C₁₋₆ alkyl;
R¹¹ is hydrogen, substituted or unsubstituted C₁₋₆ alkyl, substituted or unsubstituted heterocycle or substituted or unsubstituted C₆₋₁₀ aryl;
R¹² is hydrogen, hydroxyl, substituted or unsubstituted heterocycle, substituted or unsubstituted C₆₋₁₀ aryl or substituted or unsubstituted C₁₋₆ alkyl;
except compounds:

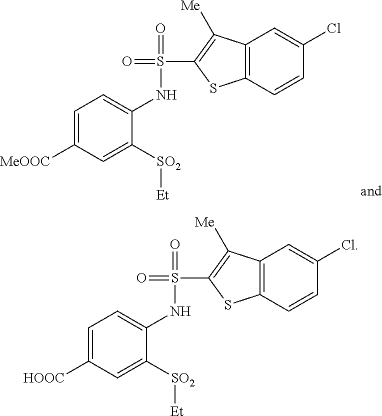 [SMILES: CCS(=O)(=O)c1cc(C(=O)OC)ccc1NS(=O)(=O)c1sc2ccc(Cl)cc2c1C ; CCS(=O)(=O)c1cc(C(=O)O)ccc1NS(=O)(=O)c1sc2ccc(Cl)cc2c1C]

### DETAILED DESCRIPTION OF THE INVENTION

Described herein are a group of novel benzothiophene sulfonamide derivatives which are potent and selective chemokine receptor modulators, as well as methods associated with the benzothiophene sulfonamide derivatives. As such, the compounds described herein are useful in treating a wide variety of disorders associated with modulation of chemokine receptors.

The term “modulator” as used herein, includes but is not limited to: receptor agonist, antagonist, inverse agonist, inverse antagonist, partial agonist, partial antagonist.

The term “alkyl”, as used herein, refers to saturated, monovalent or divalent hydrocarbon moieties having linear or branched moieties or combinations thereof and containing 1 to 6 carbon atoms. One methylene (—CH₂—) group, of the alkyl can be replaced by oxygen, sulfur, sulfoxide, nitrogen, carbonyl, carboxyl, sulfonyl, or by a divalent C₃₋₆ cycloalkyl. Hydrogen atoms on alkyl groups can be substituted by groups including, but not limited to: halogens, —OH, C₃₋₈ cycloalkyl, non-aromatic heterocycles, aromatic heterocycles, —OC₁₋₆ alkyl, -amines, —NO₂, amides, carboxylic acids, ketones, ethers, esters, aldehydes, or sulfonamides.

The term “cycloalkyl”, as used herein, refers to a monovalent or divalent group of 3 to 8 carbon atoms, derived from a saturated cyclic hydrocarbon. Cycloalkyl groups can be monocyclic or polycyclic. Cycloalkyl can be substituted by groups including, but not limited to: halogens, —OH, Cₘ cycloalkyl, non-aromatic heterocycles, aromatic heterocycles, —OC₁₋₆ alkyl, -amines, —NO₂, amides, ethers, esters, carboxylic acids, aldehydes, ketones, or sulfonamides.

The term “cycloalkenyl”, as used herein, refers to a monovalent or divalent group of 3 to 8 carbon atoms, derived from a saturated cycloalkyl having one or more double bonds. Cycloalkenyl groups can be monocyclic or polycyclic. Cycloalkenyl groups can be substituted by groups including, but not limited to: halogens, —OH, C₃₋₈ cycloalkyl, non-aromatic heterocycles, aromatic heterocycles, —OC₁₋₆ alkyl, -amines, —NO₂, amides, ethers, esters, aldehydes, ketones, carboxylic acids, sulfonamide groups.

The term “halogen”, as used herein, refers to an atom of chlorine, bromine, fluorine, or iodine.

The term “alkenyl”, as used herein, refers to a monovalent or divalent hydrocarbon radical having 2 to 6 carbon atoms, derived from a saturated alkyl, having at least one double bond. C₂₋₆ alkenyl can be in the E or Z configuration. Alkenyl groups can be substituted by C₁₋₆ alkyl.

The term “alkynyl”, as used herein, refers to a monovalent or divalent hydrocarbon radical having 2 to 6 carbon atoms, derived from a saturated alkyl, having at least one triple bond.

The term “heterocycle” as used herein, refers to a 3 to 10 membered ring, which can be aromatic or non-aromatic, saturated or unsaturated, containing at least one heteroatom selected from O or N or S or combinations of at least two thereof, interrupting the carbocyclic ring structure. The heterocyclic ring can be interrupted by a C═O; the S heteroatom can be oxidized. Heterocycles can be monocyclic or polycyclic. Heterocyclic ring moieties can be substituted by groups including, but not limited to: halogens, —OH, C₃₋₈ cycloalkyl, non-aromatic heterocycles, aromatic heterocycles, — OC₁₋₆ alkyl, -amines, —NO₂, amides, ethers, esters, aldehydes, carboxylic acids, ketones, sulfonamides groups.

The term “aryl” as used herein, refers to an organic moiety derived from an aromatic hydrocarbon consisting of a ring containing 6 to 10 carbon atoms by removal of one hydrogen. Aryl can be monocyclic or polycyclic. Aryl can be substituted by groups including, but not limited to: halogens, —OH, C₃₋₈ cycloalkyl, non-aromatic heterocycles, aromatic heterocycles, —OC₁₋₆ alkyl, -amines, —NO₂, amides, ethers, esters, carboxylic acids, ketones, aldehydes, sulfonamide groups.

The term “amide” as used herein, represents a group of formula “—C(O)NR^{x}R^{y}”, wherein R^{x} and R^{y} are the same or independently H or C₁₋₆ alkyl.

The term “ketone” as used herein, represents a group of formula “—C(O)R^{x}”, wherein R^{x} is C₁₋₆ alkyl.

The term “ester” as used herein, represents a group of formula “—C(O)OR^{x}”, wherein R^{x} is C₁₋₆ alkyl.

The term “ether” as used herein, represents a group of formula “—OR^{x}”, wherein R^{x} is C₁₋₆ alkyl.

The term “aldehyde” as used herein, represents a group of formula “—C(O)H”.

The term “sulfonamide” as used herein, represents a group of formula “—S(O)₂NR^{x}R^{y}”, wherein R^{x} and R^{y} are the same or independently H or C₁₋₆ alkyl.

The term “hydroxyl” as used herein, represents a group of formula “—OH”.

The term “amine” as used herein, represents a group of formula “NR^{x}R^{y}”, wherein R^{x} and R^{y} are the same or independently H or C₁₋₆ alkyl.

The term “carbonyl” as used herein, represents a group of formula “—C(O)—”.

The term “carboxyl” as used herein, represents a group of formula “—C(O)O-”.

The term “sulfonyl” or the term “sulfone” as used herein, represents a group of formula “—SO₂-”.

The term “sulfate” as used herein, represents a group of formula “—O—S(O)₂—O-”.

The term “carboxylic acid” as used herein, represents a group of formula “—C(O)OH”.

The term “sulfoxide” as used herein, represents a group of formula “—S(O)—”.

The term “phosphonic acid” as used herein, represents a group of formula “—P(O)(OH)₂”.

The term “phosphoric acid” as used herein, represents a group of formula “—O—P(O)(OH)₂”.

The term “sulphonic acid” as used herein, represents a group of formula “—S(O)₂OH”.

The formula “H”, as used herein, represents a hydrogen atom.

The formula “O”, as used herein, represents an oxygen atom.

The formula “N”, as used herein, represents a nitrogen atom.

The formula “S”, as used herein, represents a sulfur atom.

In some embodiments, the invention provides a compound having Formula I or a pharmaceutically acceptable salt thereof or stereoisomeric forms thereof, or the, enantiomers, diastereoisomers, tautomers, zwitterions and pharmaceutically acceptable salts thereof:

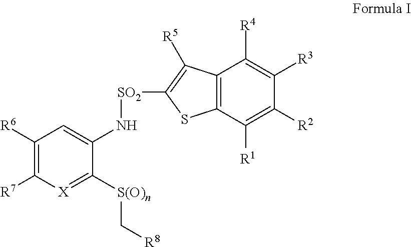

wherein:
X is N or CR;
R is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R¹ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R² is hydrogen, halogen, CN, substituted or unsubstituted C₁₋₆ alkyl, substituted or unsubstituted Cₘ cycloalkyl, OR⁹, NR¹⁰R¹¹ or COR¹²;
R³ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁴ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁵ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁶ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁷ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁸ is substituted or unsubstituted C₁₋₆ alkyl, substituted or unsubstituted C₃₋₈ cycloalkyl, substituted or unsubstituted aryl or substituted or unsubstituted heteroaryl;
n is 0, 1 or 2;
R⁹ is hydrogen or substituted or unsubstituted C₁₋₆ alkyl;
R¹⁰ is hydrogen or substituted or unsubstituted C₁₋₆ alkyl;
R¹¹ is hydrogen, substituted or unsubstituted C₁₋₆ alkyl, substituted or unsubstituted heterocycle or substituted or unsubstituted C₆₋₁₀ aryl;
R¹² is hydrogen, hydroxyl, substituted or unsubstituted heterocycle, substituted or unsubstituted C₆₋₁₀ aryl or substituted or unsubstituted C₁₋₆ alkyl;
except compounds:

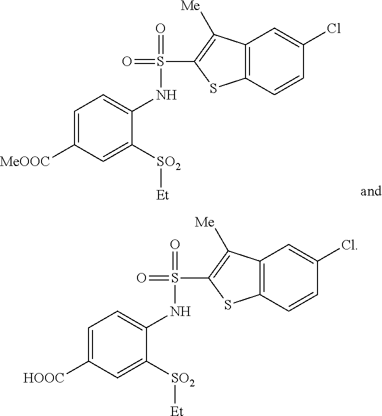 [SMILES: CCS(=O)(=O)c1cc(C(=O)OC)ccc1NS(=O)(=O)c1sc2ccc(Cl)cc2c1C ; CCS(=O)(=O)c1cc(C(=O)O)ccc1NS(=O)(=O)c1sc2ccc(Cl)cc2c1C]

In particular, in some embodiments of Formula I, X is CR, wherein R is hydrogen:

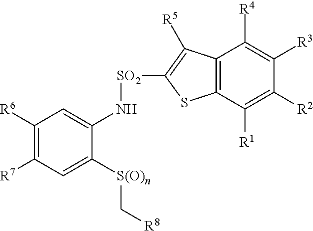

and wherein R¹ to R⁸ and n are as defined herein.

In particular, in some embodiments of Formula I, X is CR, wherein R is hydrogen, n is 0:

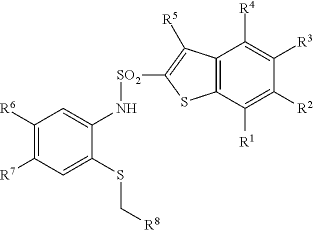

and wherein R¹ to R⁸ are as defined herein.

In some embodiments, compounds of the invention are:
methyl 2-[({2-[(1-benzothiophen-2-ylsulfonyl)amino]-4-fluorophenyl}sulfanyl)methyl]benzoate;
2-[({2-[(1-benzothiophen-2-ylsulfonyl)amino]-4-fluorophenyl}sulfanyl)methyl]benzoic acid;
N-[2-(Benzylsulfinyl)pyridin-3-yl]-1-benzothiophene-2-sulfonamide;
N-[2-(Benzylsulfonyl)pyridin-3-yl]-1-benzothiophene-2-sulfonamide;
N-[2-(Benzylsulfanyl)pyridin-3-yl]-1-benzothiophene-2-sulfonamide;
2-[({2-[(1-Benzothiophen-2-ylsulfonyl)amino]-5-methylphenyl}sulfanyl)methyl]benzoic acid;
Methyl 2-[({2-[(1-Benzothiophen-2-ylsulfonyl)amino]-5-methylphenyl}sulfanyl) methyl]benzoate;
N-(5-Chloro-2-((3-nitrobenzyl)thio)phenyl)benzo[b]thiophene-2-sulfonamide;
N-(5-Chloro-2-((3-nitrobenzyl)sulfonyl)phenyl)benzo[b]thiophene-2-sulfonamide;
N-{2-[(3-Aminobenzyl)sulfonyl]-5-chlorophenyl}-1-benzothiophene-2-sulfonamide;
2-[({2-[(1-Benzothiophen-2-ylsulfonyl)amino]-4-methylphenyl}sulfanyl)methyl]benzoic acid;
Methyl 2-[({2-[(1-benzothiophen-2-ylsulfonyl)amino]-4-methylphenyl}sulfanyl) methyl]benzoate;
2-[({2-[(1-Benzothiophen-2-ylsulfonyl)amino]-4-chlorophenyl}sulfanyl)methyl]benzoic acid; and
Methyl 2-[({2-[(1-benzothiophen-2-ylsulfonyl)amino]-4-chlorophenyl}sulfanyl) methyl]benzoate.

Some compounds of Formula I and some of their intermediates have at least one stereogenic center in their structure. This stereogenic center may be present in an R or S configuration, said R and S notation is used in correspondence with the rules described in Pure Appl. Chem. (1976), 45, 11-13.

In some embodiments, the compounds described herein can be exist as pharmaceutically acceptable salts.

The term “pharmaceutically acceptable salts” refers to salts or complexes that retain the desired biological activity of the above identified compounds and exhibit minimal or no undesired toxicological effects. The “pharmaceutically acceptable salts” according to the invention include therapeutically active, non-toxic base or acid salt forms, which the compounds of Formula I are able to form.

The acid addition salt form of a compound of Formula I that occurs in its free form as a base can be obtained by treating the free base with an appropriate acid such as an inorganic acid, for example, hydrochloric acid, hydrobromic acid, sulfuric acid, phosphoric acid, nitric acid and the like; or an organic acid such as for example, acetic, hydroxyacetic, propanoic, lactic, pyruvic, malonic, fumaric acid, maleic acid, oxalic acid, tartaric acid, succinic acid, malic acid, ascorbic acid, benzoic acid, tannic acid, pamoic acid, citric, methylsulfonic, ethanesulfonic, benzenesulfonic, formic acid and the like (Handbook of Pharmaceutical Salts, P. Heinrich Stahl & Camille G. Wermuth (Eds), Verlag Helvetica Chimica Acta-Zürich, 2002, 329-345).

The base addition salt form of a compound of Formula I that occurs in its acid form can be obtained by treating the acid with an appropriate base such as an inorganic base, for example, sodium hydroxide, magnesium hydroxide, potassium hydroxide, calcium hydroxide, ammonia and the like; or an organic base such as for example, L-Arg in ine, ethanolamine, betaine, benzathine, morpholine and the like. (Handbook of Pharmaceutical Salts, P. Heinrich Stahl& Camille G. Wermuth (Eds), Verlag Helvetica Chimica Acta-Zurich, 2002, 329-345).

With respect to the present invention reference to a compound or compounds, is intended to encompass that compound in each of its possible isomeric forms and mixtures thereof unless the particular isomeric form is referred to specifically.

Compounds according to the present invention may exist in different polymorphic forms. Although not explicitly indicated in the above formula, such forms are intended to be included within the scope of the present invention.

The compounds of the invention are indicated for use in treating or preventing conditions in which there is likely to be a component involving the chemokine receptors.

In another embodiment, there are provided pharmaceutical compositions including at least one compound of the invention in a pharmaceutically acceptable carrier.

In a further embodiment of the invention, there are provided methods for treating disorders associated with modulation of chemokine receptors. Such methods can be performed, for example, by administering to a subject in need thereof a pharmaceutical composition containing a therapeutically effective amount of at least one compound of the invention.

In particular, in some embodiments, there is provided a method of treating a disorder associated with chemokine receptor modulation, which comprises administering to a mammal in need thereof, a pharmaceutical composition comprising a therapeutically effective amount of at least one compound of Formula I

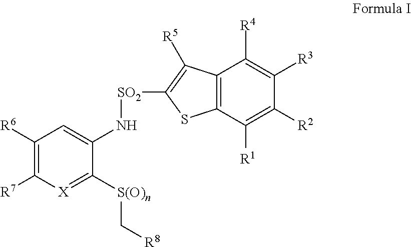

or a pharmaceutically acceptable salt thereof, wherein:
X is N or CR;
R is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R¹ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R² is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R³ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁴ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁵ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁶ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁷ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁸ is substituted or unsubstituted C₁₋₆ alkyl, substituted or unsubstituted C₃₋₈ cycloalkyl, substituted or unsubstituted aryl or substituted or unsubstituted heteroaryl;
n is 0, 1 or 2;
R⁹ is hydrogen or substituted or unsubstituted C₁₋₆ alkyl;
R¹⁰ is hydrogen or substituted or unsubstituted C₁₋₆ alkyl;
R¹¹ is hydrogen, substituted or unsubstituted C₁₋₆ alkyl, substituted or unsubstituted heterocycle or substituted or unsubstituted C₆₋₁₀ aryl;
R¹² is hydrogen, hydroxyl, substituted or unsubstituted heterocycle, substituted or unsubstituted C₆₋₁₀ aryl or substituted or unsubstituted C₁₋₆ alkyl;
except compounds:

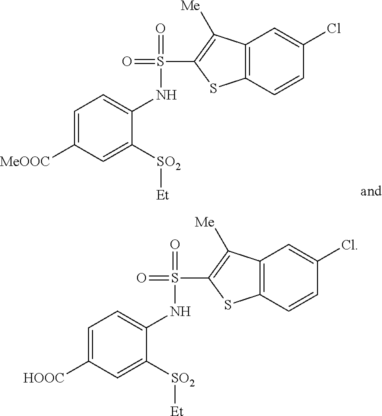 [SMILES: CCS(=O)(=O)c1cc(C(=O)OC)ccc1NS(=O)(=O)c1sc2ccc(Cl)cc2c1C ; CCS(=O)(=O)c1cc(C(=O)O)ccc1NS(=O)(=O)c1sc2ccc(Cl)cc2c1C]

These compounds are useful for the treatment of mammals, including humans, with a range of conditions and diseases that are alleviated by chemokine receptor modulation.

Therapeutic utilities of chemokine receptor modulators are skin inflammatory diseases and conditions, including, but are not limited to: rosacea (dilation of the blood vessels just under the skin), sunburn, chronic sun damage, discreet erythemas, psoriasis, atopic dermatitis, menopause-associated hot flashes, hot flashes resulting from orchiectomyatopic dermatitis, photoaging, seborrheic dermatitis, acne, allergic dermatitis, irritant dermatitis, telangiectasia (dilations of previously existing small blood vessels) of the face, rhinophyma (hypertrophy of the nose with follicular dilation), red bulbous nose, acne-like skin eruptions (may ooze or crust), burning or stinging sensation of the face, irritated and bloodshot and watery eyes, cutaneous hyperactivity with dilation of blood vessels of the skin, Lyell's syndrome, Stevens-Johnson syndrome, erythema multiforme minor, erythema multiforme major and other inflammatory skin diseases, actinic keratoses, arsenic keratoses, inflammatory and non-inflammatory acne, ichthyoses and other keratinization and hyperproliferative disorders of the skin, eczema, wound healing.

Therapeutic utilities of chemokine receptor modulators are ocular inflammatory diseases including, but not limited to, uveitis, dry eye, keratitis, allergic eye disease and conditions affecting the posterior part of the eye, such as maculopathies and retinal degeneration including non-exudative age related macular degeneration, exudative age related macular degeneration, choroidal neovascularization, diabetic retinopathy, acute macular neuroretinopathy, central serous chorioretinopathy, cystoid macular edema, and diabetic macular edema; uveitis, retinitis, and choroiditis such as acute multifocal placoid pigment epitheliopathy, Behcet's disease, birdshot retinochoroidopathy, infectious (syphilis, lyme, tuberculosis, toxoplasmosis), intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi- and Harada syndrome; vasuclar diseases/exudative diseases such as retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angiitis, sickle cell retinopathy and other hemoglobinopathies, angioid streaks, familial exudative vitreoretinopathy, and Eales disease; traumatic/surgical conditions such as sympathetic ophthalmia, uveitic retinal disease, retinal detachment, trauma, conditions caused by laser, conditions caused by photodynamic therapy, photocoagulation, hypoperfusion during surgery, radiation retinopathy, and bone marrow transplant retinopathy; proliferative disorders such as proliferative vitreal retinopathy and epiretinal membranes, and proliferative diabetic retinopathy; infectious disorders such as ocular histoplasmosis, ocular toxocariasis, presumed ocular histoplasmosis syndrome (POHS), endophthalmitis, toxoplasmosis, retinal diseases associated with HIV infection, choroidal disease associate with HIV infection, uveitic disease associate with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; and miscellaneous other diseases affecting the posterior part of the eye such as punctate inner choroidopathy, acute posterior multifocal placoid pigment epitheliopathy, myopic retinal degeneration, and acute retinal pigement epitheliitis.

In still another embodiment of the invention, there are provided methods for treating disorders associated with modulation of chemokine receptors. Such methods can be performed, for example, by administering to a subject in need thereof a therapeutically effective amount of at least one compound of the invention, or any combination thereof, or pharmaceutically acceptable salts, individual enantiomers, and diastereomers thereof.

The present invention concerns the use of a compound of Formula I or a pharmaceutically acceptable salt thereof, for the manufacture of a medicament for the treatment of ocular inflammatory diseases including, but not limited to, uveitis, dry eye, Keratitis, allergic eye disease and conditions affecting the posterior part of the eye, such as maculopathies and retinal degeneration including non-exudative age related macular degeneration, exudative age related macular degeneration, choroidal neovascularization, diabetic retinopathy, acute macular neuroretinopathy, central serous chorioretinopathy, cystoid macular edema, and diabetic macular edema; uveitis, retinitis, and choroiditis such as acute multifocal placoid pigment epitheliopathy, Behcet's disease, birdshot retinochoroidopathy, infectious (syphilis, lyme, tuberculosis, toxoplasmosis), intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi- and Harada syndrome; vasuclar diseases/exudative diseases such as retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angiitis, sickle cell retinopathy and other hemoglobinopathies, angioid streaks, familial exudative vitreoretinopathy, and Eales disease; traumatic/surgical conditions such as sympathetic ophthalmia, uveitic retinal disease, retinal detachment, trauma, conditions caused by laser, conditions caused by photodynamic therapy, photocoagulation, hypoperfusion during surgery, radiation retinopathy, and bone marrow transplant retinopathy; proliferative disorders such as proliferative vitreal retinopathy and epiretinal membranes, and proliferative diabetic retinopathy; infectious disorders such as ocular histoplasmosis, ocular toxocariasis, presumed ocular histoplasmosis syndrome (PONS), endophthalmitis, toxoplasmosis, retinal diseases associated with HIV infection, choroidal disease associate with HIV infection, uveitic disease associate with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; and miscellaneous other diseases affecting the posterior part of the eye such as punctate inner choroidopathy, acute posterior multifocal placoid pigment epitheliopathy, myopic retinal degeneration, and acute retinal pigement epitheliitis.

The actual amount of the compound to be administered in any given case will be determined by a physician taking into account the relevant circumstances, such as the severity of the condition, the age and weight of the patient, the patient's general physical condition, the cause of the condition, and the route of administration.

The patient will be administered the compound orally in any acceptable form, such as a tablet, liquid, capsule, powder and the like, or other routes may be desirable or necessary, particularly if the patient suffers from nausea. Such other routes may include, without exception, transdermal, parenteral, subcutaneous, intranasal, via an implant stent, intrathecal, intravitreal, topical to the eye, back to the eye, intramuscular, intravenous, and intrarectal modes of delivery. Additionally, the formulations may be designed to delay release of the active compound over a given period of time, or to carefully control the amount of drug released at a given time during the course of therapy.

In another embodiment of the invention, there are provided pharmaceutical compositions including at least one compound of the invention in a pharmaceutically acceptable carrier thereof. The phrase “pharmaceutically acceptable” means the carrier, diluent or excipient must be compatible with the other ingredients of the formulation and not deleterious to the recipient thereof.

Pharmaceutical compositions of the present invention can be used in the form of a solid, a solution, an emulsion, a dispersion, a patch, a micelle, a liposome, and the like, wherein the resulting composition contains one or more compounds of the present invention, as an active ingredient, in admixture with an organic or inorganic carrier or excipient suitable for enteral or parenteral applications. Invention compounds may be combined, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. Invention compounds are included in the pharmaceutical composition in an amount sufficient to produce the desired effect upon the process or disease condition.

Pharmaceutical compositions containing invention compounds may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of a sweetening agent such as sucrose, lactose, or saccharin, flavoring agents such as peppermint, oil of wintergreen or cherry, coloring agents and preserving agents in order to provide pharmaceutically elegant and palatable preparations. Tablets containing invention compounds in admixture with non-toxic pharmaceutically acceptable excipients may also be manufactured by known methods. The excipients used may be, for example, (1) inert diluents such as calcium carbonate, lactose, calcium phosphate or sodium phosphate; (2) granulating and disintegrating agents such as corn starch, potato starch or alginic acid; (3) binding agents such as gum tragacanth, corn starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

In some cases, formulations for oral use may be in the form of hard gelatin capsules wherein the invention compounds are mixed with an inert solid diluent, for example, calcium carbonate, calcium phosphate or kaolin. They may also be in the form of soft gelatin capsules wherein the invention compounds are mixed with water or an oil medium, for example, peanut oil, liquid paraffin or olive oil.

The pharmaceutical compositions may be in the form of a sterile injectable suspension. This suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. Buffers, preservatives, antioxidants, and the like can be incorporated as required.

Invention compounds and their pharmaceutically-acceptable salts may be administered through different routes, including but not limited to topical eye drops, direct injection, application at the back of the eye or formulations that may further enhance the long duration of actions such as a slow releasing pellet, suspension, gel, or sustained delivery devices such as any suitable drug delivery system (DDS) known in the art. While topical administration is preferred, this compound may also be used in an intraocular implant as described in U.S. Pat. No. 7,931,909.

Pharmaceutical compositions containing invention compounds may be in a form suitable for topical use, for example, as oily suspensions, as solutions or suspensions in aqueous liquids or nonaqueous liquids, or as oil-in-water or water-in-oil liquid emulsions. Pharmaceutical compositions may be prepared by combining a therapeutically effective amount of at least one compound according to the present invention, or a pharmaceutically acceptable salt thereof, as an active ingredient with conventional ophthalmically acceptable pharmaceutical excipients and by preparation of unit dosage suitable for topical ocular use. The therapeutically efficient amount typically is between about 0.0001 and about 5% (w/v), preferably about 0.001 to about 2.0% (w/v) in liquid formulations.

For ophthalmic application, preferably solutions are prepared using a physiological saline solution as a major vehicle. The pH of such ophthalmic solutions should preferably be maintained between 4.5 and 8.0 with an appropriate buffer system, a neutral pH being preferred but not essential. The formulations may also contain conventional pharmaceutically acceptable preservatives, stabilizers and surfactants. Preferred preservatives that may be used in the pharmaceutical compositions of the present invention include, but are not limited to, benzalkonium chloride, chlorobutanol, thimerosal, phenylmercuric acetate and phenylmercuric nitrate. A preferred surfactant is, for example, Tween 80. Likewise, various preferred vehicles may be used in the ophthalmic preparations of the present invention. These vehicles include, but are not limited to, polyvinyl alcohol, povidone, hydroxypropyl methyl cellulose, poloxamers, carboxymethyl cellulose, hydroxyethyl cellulose cyclodextrin and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. Accordingly, buffers include acetate buffers, citrate buffers, phosphate buffers and borate buffers. Acids or bases may be used to adjust the pH of these formulations as needed.

In a similar manner an ophthalmically acceptable antioxidant for use in the present invention includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. The preferred chelating agent is edentate disodium, although other chelating agents may also be used in place of or in conjunction with it.

The ingredients are usually used in the following amounts:

| Ingredient | Amount (% w/v) |
|---|---|
| active ingredient | about 0.001-5 |
| preservative | 0-0.10 |
| vehicle | 0-40 |
| tonicity adjustor | 1-10 |
| buffer | 0.01-10 |
| pH adjustor | q.s. pH 4.5-7.5 |
| antioxidant | as needed |
| surfactant | as needed |
| purified water | as needed to make 100% |

The actual dose of the active compounds of the present invention depends on the specific compound, and on the condition to be treated; the selection of the appropriate dose is well within the knowledge of the skilled artisan.

The ophthalmic formulations of the present invention are conveniently packaged in forms suitable for metered application, such as in containers equipped with a dropper, to facilitate application to the eye. Containers suitable for dropwise application are usually made of suitable inert, non-toxic plastic material, and generally contain between about 0.5 and about 15 ml solution. One package may contain one or more unit doses. Especially preservative-free solutions are often formulated in non-resealable containers containing up to about ten, preferably up to about five units doses, where a typical unit dose is from one to about 8 drops, preferably one to about 3 drops. The volume of one drop usually is about 20-35 μl (microliter).

Invention compounds may also be administered in the form of suppositories for rectal administration of the drug. These compositions may be prepared by mixing the invention compounds with a suitable non-irritating excipient, such as cocoa butter, synthetic glyceride esters of polyethylene glycols, which are solid at ordinary temperatures, but liquefy and/or dissolve in the rectal cavity to release the drug.

Since individual subjects may present a wide variation in severity of symptoms and each drug has its unique therapeutic characteristics, the precise mode of administration and dosage employed for each subject is left to the discretion of the practitioner.

The compounds and pharmaceutical compositions described herein are useful as medicaments in mammals, including humans, for treatment of diseases and/or alleviations of conditions which are responsive to treatment by agonists or functional antagonists of chemokine receptors. Thus, in further embodiments of the invention, there are provided methods for treating a disorder associated with modulation of chemokine receptors. Such methods can be performed, for example, by administering to a subject in need thereof a pharmaceutical composition containing a therapeutically effective amount of at least one invention compound. As used herein, the term “therapeutically effective amount” means the amount of the pharmaceutical composition that will elicit the biological or medical response of a subject in need thereof that is being sought by the researcher, veterinarian, medical doctor or other clinician. In some embodiments, the subject in need thereof is a mammal. In some embodiments, the mammal is human.

The present invention concerns also processes for preparing the compounds of Formula I. The compounds of formula I according to the invention can be prepared analogously to conventional methods as understood by the person skilled in the art of synthetic organic chemistry. The described benzothiophene-2-sulfonamide derivatives were prepared by methods as shown in Scheme 1. Those skilled in the art will be able to routinely modify and/or adapt Scheme 1 to synthesize any compounds of the invention covered by Formula I.

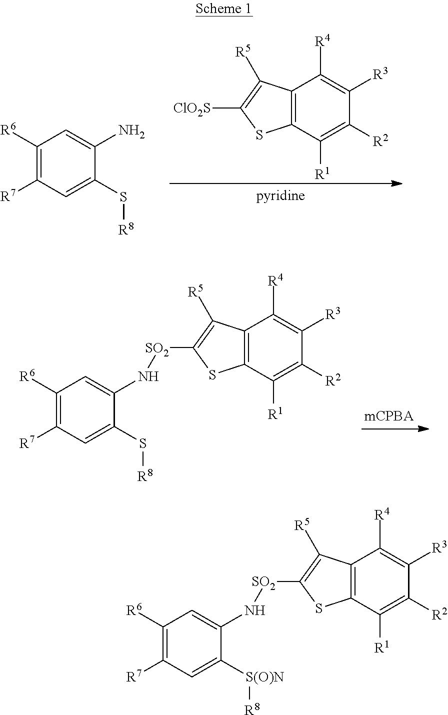

It is to be understood that both the foregoing general description and the following detailed description are exemplary and explanatory only and are not restrictive of the invention claimed. As used herein, the use of the singular includes the plural unless specifically stated otherwise.

It will be readily apparent to those skilled in the art that some of the compounds of the invention may contain one or more asymmetric centers, such that the compounds may exist in enantiomeric as well as in diastereomeric forms. Unless it is specifically noted otherwise, the scope of the present invention includes all enantiomers, diastereomers and racemic mixtures. Some of the compounds of the invention may form salts with pharmaceutically acceptable acids or bases, and such pharmaceutically acceptable salts of the compounds described herein are also within the scope of the invention.

The present invention includes all pharmaceutically acceptable isotopically enriched compounds. Any compound of the invention may contain one or more isotopic atoms enriched or different than the natural ratio such as deuterium ²H (or D) in place of protium ¹H (or H) or use of ¹³ C enriched material in place of ¹²C and the like. Similar substitutions can be employed for N, O and S. The use of isotopes may assist in analytical as well as therapeutic aspects of the invention. For example, use of deuterium may increase the in vivo half-life by altering the metabolism (rate) of the compounds of the invention. These compounds can be prepared in accord with the preparations described by use of isotopically enriched reagents.

As will be evident to those skilled in the art, individual isomeric forms can be obtained by separation of mixtures thereof in conventional manner. For example, in the case of diasteroisomeric isomers, chromatographic separation may be employed.

Compound names were generated with ACDLabs version 12.5 and some intermediates' and reagents' names used in the examples were generated with software such as Chem Bio Draw Ultra version 12.0 or Auto Nom 2000 from MDL ISIS Draw 2.5 SP1. In general, characterization of the compounds is performed according to the following methods:

NMR spectra are recorded on Varian 600 or Varian 300, in the indicated solvent at ambient temperature; chemical shifts in [ppm], coupling constants in [Hz].

All the reagents, solvents, catalysts for which the synthesis is not described are purchased from chemical vendors such as Sigma Aldrich, Fluka, Bio-Blocks, Combi-blocks, TCI, VWR, Lancaster, Oakwood, Trans World Chemical, Alfa, Fisher, Maybridge, Frontier, Matrix, Ukrorgsynth, Toronto, Ryan Scientific, SiliCycle, Anaspec, Syn Chem, Chem-Impex, MIC-scientific, Ltd; however some known intermediates were prepared according to published procedures. Solvents were purchased from commercial sources in appropriate quality and used as received. Air and/or moisture-sensitive reactions were run under an Ar- or N₂-atmosphere.

Usually the compounds of the invention were purified by chromatography: CombiFlash Companion and RediSep Rf silica gel 60 (0.04-0.063 mm); Preparative thin layer chromatography (PTLC): Analtech (silica gel 60 F₂₅₄, 500 or 1000 μm).

The following abbreviations are used in the examples:
CH₂Cl₂ dichloromethane
NaOH sodium hydroxide
MeOH methanol
CD₃OD deuterated methanol
HCl hydrochloric acid
DMF dimethylformamide
EtOAc ethyl acetate
CDCl₃ deuterated chloroform
DMSO-d₆ deuterated dimethyl sulfoxide
K₂CO₃ potassium carbonate
Na₂SO₄ sodium sulfate
NH₄Cl ammonium chloride
Et₂O diethylether
Na₂S.9H₂O Sodium sulfide nonahydrate

### Zn Zinc

### SYNTHETIC EXAMPLES

### Example 1

### Intermediate 1

### Methyl 2-(((2-Amino-4-chlorophenyl)thio)methyl)benzoate

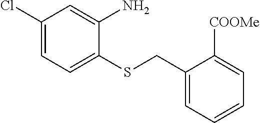 [SMILES: COC(=O)c1ccccc1CSc1ccc(Cl)cc1N]

A mixture of 2-amino-4-chlorobenzenethiol (3.5 g, 21.9 mmol), methyl 2-(bromomethyl)benzoate (5.0 g, 21.9 mmol) and K₂CO₃ (15 g, 109.7 mmol) in DMF (50 ml) was stirred at room temperature overnight. The reaction mixture was poured into water (50 ml) and extracted with ethyl acetate (2×50 ml). The organic layer was washed with brine, dried over Na₂SO₄, concentrated in vacuo. The crude was purified by column chromatography on silica gel (0˜30% ethyl acetate in hexane) to yield the title compound as a solid (5.25 g, 78%).

¹H NMR (600 MHz, CDCl₃) δ 7.91 (dd, J=1.47, 7.63 Hz, 1H), 7.25-7.43 (m, 2H), 6.92-7.06 (m, 2H), 6.67 (d, J=2.05 Hz, 1H), 6.52 (dd, J=2.05, 8.22 Hz, 1H), 4.27 (s, 2H), 3.88 (s, 3H).

### Example 2

### Intermediate 2

### Methyl 2-(((4-Methyl-2-nitrophenyl)thio)methyl)benzoate

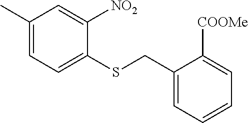 [SMILES: COC(=O)c1ccccc1CSc1ccc(C)cc1[N+](=O)[O-]]

To a solution of 1-fluoro-4-methyl-2-nitrobenzene (1.13 g, 7.28 mmol) in DMF (20 ml) was added Na₂S.9H₂O (1.75 g, 7.28 mmol) and the reaction was stirred at room temperature overnight. To this crude mixture was added methyl 2-(bromomethyl) benzoate (1.7 g, 7.28 mmol) and K₂CO₃ (5 g, 36.4 mmol) and the reaction was further stirred at room temperature for 24 hours. The reaction mixture was poured into water (50 ml) and extracted with ethyl acetate (2×50 ml). The combined organic layer was washed with brine, dried over Na₂SO₄, and concentrated in vacuo. The crude was purified by column chromatography on silica gel (0˜30% ethyl acetate in hexane) to yield the title compound as a solid (460 mg, 20%).

¹H NMR (600 MHz, CDCl₃) δ 7.97 (td, J=1.47, 3.81 Hz, 2H), 7.42-7.52 (m, 2H), 7.28-7.38 (m, 3H), 4.65 (s, 2H), 3.89 (s, 3H), 2.38 (s, 3H).

### Example 3

### Intermediate 3

### Methyl 2-(((2-Amino-4-methylphenyl)thio)methyl)benzoate

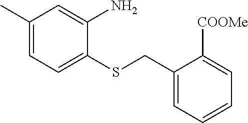 [SMILES: COC(=O)c1ccccc1CSc1ccc(C)cc1N]

### Intermediate 2

(60 mg, 1.5 mmol) was dissolved in MeOH (20 ml). Zn dust (1.9 g, 29 mmol) and NH₄Cl (1 ml) was added to the solution. After the mixture was stirred for 30 min at room temperature, the solid was filtered and the filtrate was concentrated in vacu. The crude product was used directly without further purification (352 mg, 84%).

¹H NMR (600 MHz, CDCl₃) δ 7.90 (dd, J=1.47, 7.63 Hz, 1H), 7.21-7.35 (m, 3H), 6.94-7.03 (m, 2H), 6.50-6.50 (m, 1H), 6.40 (dd, J=1.17, 7.63 Hz, 1H), 4.27 (s, 2H), 3.88 (s, 3H), 2.23 (s, 3H).

### Example 4

### Intermediate 4

### 5-Chloro-2-((3-Nitrobenzyl)thio)aniline hydrochloride

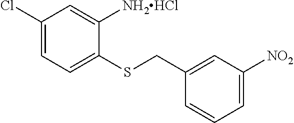

A solution of 2-amino-4-chlorobenzenethiol (6.4 g, 40 mmol), 1-(bromomethyl)-3-nitrobenzene (8.7 g, 40 mmol), and 4 M NaOH (15 ml, 60 mmol) in MeOH (100 ml) was stirred at room temperature for 4 hours and was concentrated. The residue was dissolved in EtOAc, washed successively with 1 M NaOH, brine, 1 M HCl, and brine (×2), dried over Na₂SO₄, and concentrated. The crude product was dissolved in minimal amount of MeOH, diluted with Et₂O, treated with 2 M HCl in Et₂O. The resulting solid was filtered, rinsed with Et₂O (×3) to yield the title compound (10 g, 75%). ¹H NMR (METHANOL-d₄) δ: 8.08-8.17 (m, 2H), 7.60-7.68 (m, 1H), 7.49-7.57 (m, 1H), 7.36 (d, J=8.5 Hz, 1H), 7.32 (d, J=2.3 Hz, 1H), 7.16-7.23 (m, 1H), 4.25 (s, 2H).

### Example 5

### Compound 1

### Methyl 2-[({2-[(1-Benzothiophen-2-ylsulfonyflamino]-4-chlorophenyl}sulfanyl)methyl]benzoate

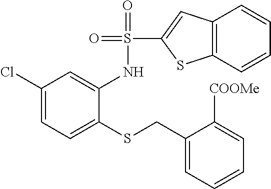 [SMILES: COC(=O)c1ccccc1CSc1ccc(Cl)cc1NS(=O)(=O)c1cc2ccccc2s1]

A mixture of methyl 2-(((2-amino-4-chlorophenyl)thio)methyl)benzoate (Intermediate 1, 300 mg, 0.97 mmol) and benzo[b]thiophene-2-sulfonyl chloride (226 mg, 0.97 mmol) in pyridine (4 ml) was heated at 100° C. overnight. Pyridine was removed under reduced pressure and the residue was loaded on silica gel column directly and the title compound was isolated with 20% EtOAc in hexane (218 mg, 44%).

¹H NMR (600 MHz, CDCl₃) δ 8.34 (s, 1H), 7.88 (d, J=0.59 Hz, 1H), 7.84 (s, 1H), 7.77-7.81 (m, 1H), 7.75 (d, J=2.35 Hz, 1H), 7.38-7.48 (m, 2H), 7.28-7.32 (m, 2H), 7.12 (d, J=8.22 Hz, 1H), 6.92 (dd, J=2.05, 8.22 Hz, 1H), 6.77-6.82 (m, 1H), 4.16 (s, 2H), 3.94 (s, 3H).

### Example 6

### Compound 2

### 2-[({2-[(1-Benzothiophen-2-ylsulfonyl)amino]-4-chlorophenyl}sulfanyl) methyl]benzoic acid

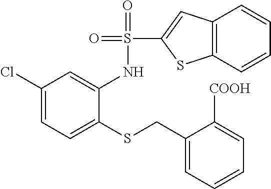 [SMILES: O=C(O)c1ccccc1CSc1ccc(Cl)cc1NS(=O)(=O)c1cc2ccccc2s1]

To methyl 2-[({2-[(1-benzothiophen-2-ylsulfonyl)amino]-4-chlorophenyl}sulfanyl) methyl]benzoate (Compound 1, 193 mg, 0.38 mmol) in MeOH (5 ml) was added 5M NaOH (2 ml) and the reaction was stirred at room temperature for 16 hours. The mixture was acidified with 10% HCl, extracted with EtOAc (×2). The combined organic layer was washed with brine, dried over Na₂SO₄, and concentrated in vacuo. The crude product was recrystallized from minimal amount of MeOH and CH₂Cl₂ to yield the title compound (187 mg, 99%).

¹H NMR (600 MHz, CD₃OD) δ 7.90 (dd, J=1.47, 7.63 Hz, 1H), 7.81-7.87 (m, 3H), 7.62 (s, 1H), 7.33-7.47 (m, 2H), 7.14-7.26 (m, 2H), 7.10 (d, J=8.22 Hz, 1H), 6.94-7.02 (m, 1H), 6.65 (dd, J=1.03, 7.48 Hz, 1H), 4.11 (s, 2H).

### Example 7

### Compound 3

Methyl 2-[({2-[(1-Benzothiophen-2-ylsulfonyl)amino]-4-methylphenyl}sulfanyl)methyl]benzoate

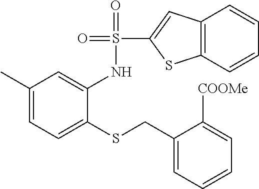 [SMILES: COC(=O)c1ccccc1CSc1ccc(C)cc1NS(=O)(=O)c1cc2ccccc2s1]

A mixture of methyl 2-(((2-amino-4-methylphenyl)thio)methyl)benzoate (Intermediate 3, 352 mg, 1.23 mmol) and benzo[b]thiophene-2-sulfonyl chloride (284 mg, 1.23 mmol) in pyridine (5 ml) was heated at 100° C. overnight. Pyridine was removed under reduced pressure and the residue was purified by flash column chromatography (20% EtOAc in hexane) to yield the title compound (430 mg, 73%).

¹H NMR (600 MHz, CD₃OD) δ 7.84-7.91 (m, 3H), 7.81 (s, 1H), 7.38-7.52 (m, 3H), 7.25 (dtd, J=1.61, 7.43, 18.74 Hz, 2H), 7.00 (s, 1H), 6.83 (d, J=8.22 Hz, 1H), 6.72 (d, J=7.63 Hz, 1H), 4.07 (s, 2H), 3.87 (s, 3H), 2.31 (s, 3H).

### Example 8

### Compound 4

### 2-[({2-[(1-Benzothiophen-2-ylsulfonyl)amino]-4-methylphenyl}sulfanyl)methyl]benzoic acid

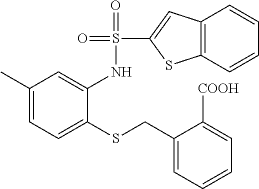 [SMILES: Cc1ccc(SCc2ccccc2C(=O)O)c(NS(=O)(=O)c2cc3ccccc3s2)c1]

To methyl 2-[({2-[(1-benzothiophen-2-ylsulfonyl)amino]-4-methylphenyl}sulfanyl) methyl]benzoate (Compound 2, 347 mg, 0.72 mmol) in MeOH (5 ml) was added 5M NaOH (2 ml) and the reaction was stirred at 80° C. for 3 hours. The mixture was acidified with 10% HCl, extracted with EtOAc (×2). The combined organic layer was washed with brine, dried over Na₂SO₄, and concentrated in vacuo. The crude product was recrystallized from minimal amount of MeOH and CH₂Cl₂ to yield the title compound (330 mg, 98%).

¹H NMR (600 MHz, CDCl₃) δ 8.38 (br. s., 1H), 8.06-8.12 (m, 1H), 7.84 (s, 1H), 7.79-7.82 (m, 1H), 7.75 (d, J=8.22 Hz, 1H), 7.56 (d, J=0.88 Hz, 1H), 7.29-7.44 (m, 4H), 7.11 (dd, J=2.49, 7.78 Hz, 1H), 6.85 (ddd, J=3.37, 3.59, 5.36 Hz, 1H), 6.73-6.80 (m, 1H), 4.16 (s, 2H), 2.33 (s, 3H).

### Example 9

### Compound 5

### N-(5-Chloro-2-((3-nitrobenzyl)thio)phenyl)benzo[b]thiophene-2-sulfonamide

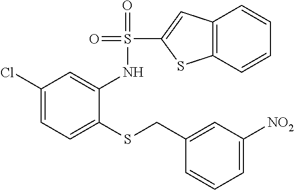 [SMILES: O=[N+]([O-])c1cccc(CSc2ccc(Cl)cc2NS(=O)(=O)c2cc3ccccc3s2)c1]

A solution of Intermediate 4 (765 mg, 2.3 mmol) and benzo[b]thiophene-2-sulfonyl chloride (537 mg, 2.3 mmol) in pyridine (5 ml) was stirred at room temperature for 16 hours and was concentrated. The residue was dissolved in EtOAc, washed successively with 1M HCl and brine, dried over Na₂SO₄, and concentrated. The crude product was purified by column chromatography on silica gel (0→30% EtOAc-hexane) to yield the title compound (0.88 g, 78%).

¹H NMR (CDCl₃) δ: 8.02-8.06 (m, 1H), 7.92-7.96 (m, 2H), 7.89 (dt, J=8.2, 0.9 Hz, 1H), 7.82-7.85 (m, 1H), 7.75-7.78 (m, 2H), 7.44-7.51 (m, 2H), 7.35 (t, J=7.9 Hz, 1H), 7.19 (dt, J=7.6, 1.3 Hz, 1H), 7.03 (d, J=8.2 Hz, 1H), 6.93 (dd, J=8.4, 2.2 Hz, 1H), 3.73 (s, 2H).

### Example 10

### Compound 6

### N-(5-Chloro-2-((3-nitrobenzyl)sulfonyl)phenyl)benzo[b]thiophene-2-sulfonamide

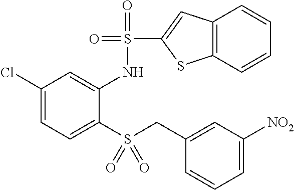 [SMILES: O=[N+]([O-])c1cccc(CS(=O)(=O)c2ccc(Cl)cc2NS(=O)(=O)c2cc3ccccc3s2)c1]

A solution of Compound 5 (0.40 g, 0.81 mmol) and 3-chlorobenzoperoxoic acid (0.39 g, 1.6 mmol) in CH₂Cl₂ (4 ml) was stirred at room temperature for 1 hour and was quenched with aqueous NaHSO₃, stirred for 10 minutes, and was extracted with EtOAc. The organic layer was separated and washed successively with aqueous Na₂CO₃ and brine, dried over Na₂SO₄, and concentrated. The crude product was dissolved in minimal amount of hot acetone, triturated with Et₂O. The resulting solid was filtered, rinsed with Et₂O to yield the title compound (0.35 g, 81%).

¹H NMR (CD₃OD) δ: 8.13 (t, J=1.8 Hz, 1H), 8.06 (ddd, J=8.2, 2.3, 0.9 Hz, 1H), 7.86 (d, J=0.9 Hz, 1H), 7.80-7.85 (m, 2H), 7.65 (d, J=2.1 Hz, 1H), 7.52 (d, J=7.6 Hz, 1H), 7.35-7.40 (m, 3H), 7.30 (t, J=7.9 Hz, 1H), 6.59 (dd, J=8.7, 1.9 Hz, 1H), 5.15 (s, 2H).

### Example 11

### Compound 7

### N-{2-[(3-Aminobenzyl)sulfonyl]-5-chlorophenyl}-1-benzothiophene-2-sulfonamide

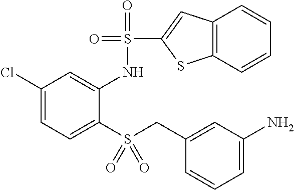 [SMILES: Nc1cccc(CS(=O)(=O)c2ccc(Cl)cc2NS(=O)(=O)c2cc3ccccc3s2)c1]

To a mixture of Compound 6 (0.34 g, 0.65 mmol) in MeOH (10 ml) and saturated aqueous NH₄Cl (5 ml) was added zinc dust (1.05 g, 16.2 mmol). The reaction was stirred at room temperature for 30 minutes, diluted with EtOAc and was filtered. The aqueous layer was separated, extracted with EtOAc, and the combined organic layer was washed with brine, dried over Na₂SO₄, and concentrated. The crude product was purified by column chromatography on silica gel (0->50% EtOAc-hexane) to yield the title compound (0.26 g, 81%).

¹H NMR (CDCl₃) δ: 8.01 (s, 1H), 7.91 (d, J=7.6 Hz, 1H), 7.84 (d, J=7.9 Hz, 1H), 7.80 (d, J=2.1 Hz, 1H), 7.43-7.54 (m, 2H), 7.39 (d, J=8.5 Hz, 1H), 7.03 (d, J=7.9 Hz, 1H), 6.96 (t, J=7.6 Hz, 1H), 6.60 (d, J=7.6 Hz, 1H), 6.31 (br. s., 1H), 6.21 (d, J=7.6 Hz, 1H), 4.15 (s, 2H).

### Example 12

### Compound 8

### Methyl 2-[({2-[(1-Benzothiophen-2-ylsulfonyl)amino]-5-methylphenyl}sulfanyl)methyl]benzoate

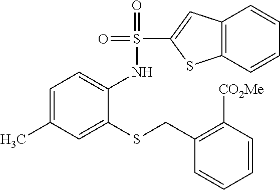 [SMILES: COC(=O)c1ccccc1CSc1cc(C)ccc1NS(=O)(=O)c1cc2ccccc2s1]

A solution of methyl 2-(((2-amino-5-methylphenyl)thio)methyl)benzoate (CAS#875895-83-3, 290 mg, 1.0 mmol) and benzo[b]thiophene-2-sulfonyl chloride (233 mg, 1.0 mmol) in pyridine (2.5 ml) was stirred at room temperature for 16 hours and was concentrated. The residue was dissolved in EtOAc, washed successively with 1M HCl and brine, dried over Na₂SO₄, and concentrated. The crude product was purified by column chromatography on silica gel (0->25% EtOAc-hexane) to yield the title compound (425 mg, 88%).

¹H NMR (CDCl₃) δ: 8.14 (s, 1H), 7.94-7.97 (m, 1H), 7.77-7.80 (m, 2H), 7.74-7.77 (m, 1H), 7.61 (d, J=8.2 Hz, 1H), 7.35-7.43 (m, 2H), 7.25-7.31 (m, 2H), 7.09-7.12 (m, 1H), 6.98-7.00 (m, 1H), 6.77-6.81 (m, 1H), 4.15 (s, 2H), 3.93 (s, 3H), 2.17 (s, 3H).

### Example 13

### Compound 9

### 2-[({2-[(1-Benzothiophen-2-ylsulfonyl)amino]-5-methylphenyl}sulfanyl)methyl]benzoic acid

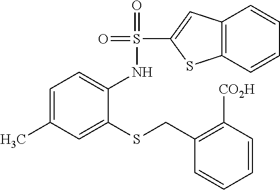 [SMILES: Cc1ccc(NS(=O)(=O)c2cc3ccccc3s2)c(SCc2ccccc2C(=O)O)c1]

A solution of Compound 8 (393 mg, 0.814 mmoL) in MeOH (20 ml) and 4 M NaOH (5.1 ml, 20.3 mmol) was stirred at 45° C. for 2 hours, cooled to 0° C., acidified with 6 M HCl, and concentrated. The crude was diluted with EtOAc, extracted with brine, dried over Na₂SO₄, and concentrated to yield the title compound (380 mg, 99%). ¹H NMR (METHANOL-d₄) δ: 7.90 (dd, J=7.8, 1.3 Hz, 1H), 7.85-7.88 (m, 2H), 7.77 (d, J=0.6 Hz, 1H), 7.39-7.48 (m, 3H), 7.25 (td, J=7.6, 1.3 Hz, 1H), 7.17-7.21 (m, 1H), 7.07-7.10 (m, 1H), 6.93 (d, J=1.2 Hz, 1H), 6.64 (dd, J=7.6, 1.2 Hz, 1H), 4.09 (s, 2H), 2.16 (s, 3H).

### Example 14

### Compound 10

### N-[2-(Benzylsulfanyl)pyridin-3-yl]-1-benzothiophene-2-sulfonamide

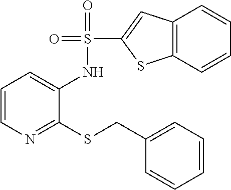 [SMILES: O=S(=O)(Nc1cccnc1SCc1ccccc1)c1cc2ccccc2s1]

A solution of 2-(benzylthio)pyridin-3-amine (CAS#69212-32-4, 243 mg, 1.13 mmol) and benzo[b]thiophene-2-sulfonyl chloride (262 mg, 1.13 mmol) in pyridine (3 ml) was stirred at room temperature for 40 hours and was concentrated. The residue was dissolved in EtOAc, washed successively with saturated aqueous NH₄Cl and brine, dried over Na₂SO₄, and concentrated. The crude product was purified by column chromatography on silica gel (0→30% EtOAc-hexane) to yield the title compound (230 mg, 49%).

¹H NMR (CDCl₃) δ: 8.33 (dd, J=4.8, 1.6 Hz, 1H), 7.86 (dd, J=8.1, 1.6 Hz, 1H), 7.78-7.83 (m, 2H), 7.77 (d, J=0.6 Hz, 1H), 7.43-7.51 (m, 2H), 7.14-7.18 (m, 3H), 7.11 (dd, J=8.1, 4.8 Hz, 1H), 7.06-7.09 (m, 2H), 6.89 (br. s., 1H), 4.26 (s, 2H).

### Example 15

### Compound 11

### N-[2-(Benzylsulfonyl)pyridin-3-yl]-1-benzothiophene-2-sulfonamide

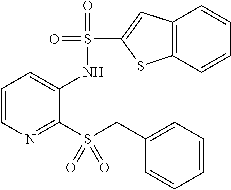 [SMILES: O=S(=O)(Cc1ccccc1)c1ncccc1NS(=O)(=O)c1cc2ccccc2s1]

and

### Compound 12

### N-[2-(Benzylsulfinyl)pyridin-3-yl]-1-benzothiophene-2-sulfonamide

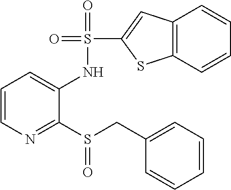 [SMILES: O=S(Cc1ccccc1)c1ncccc1NS(=O)(=O)c1cc2ccccc2s1]

A solution of Compound 10 (190 mg, 0.46 mmol) and 3-chlorobenzoperoxoic acid (120 mg, 0.692 mmol) in CH₂Cl₂ (2 ml) was stirred at room temperature for 2 hours and was directly loaded on Celite, dried, and purified by column chromatography on silica gel (0→100% EtOAc-hexane) to yield the title compounds (49 mg, 24%, and 50 mg, 25% respectively).

Compound 11 ¹H NMR (DMSO-d₆) δ: 8.39 (br. s., 1H), 8.04 (d, J=7.9 Hz, 2H), 7.98 (br. s., 2H), 7.64 (br. s., 1H), 7.42-7.54 (m, 2H), 7.15-7.22 (m, 3H), 7.10 (dd, J=7.2, 1.9 Hz, 2H), 4.82 (br. s., 2H);

Compound 12 ¹H NMR (acetone-d₆) δ: 8.21 (br. s., 1H), 7.73-8.10 (m, 4H), 7.30-7.54 (m, 3H), 6.91-7.28 (m, 5H), 4.53 (br. s., 1H), 4.14 (br. s., 1H).

### Example 16

### Intermediate 5

### methyl 2-(((2-amino-4-fluorophenyl)thio)methyl)benzoate

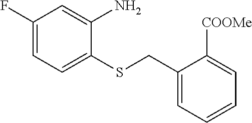 [SMILES: COC(=O)c1ccccc1CSc1ccc(F)cc1N]

A mixture of 2-amino-4-fluorobenzenethiol (0.96 g, 6.0 mmol), methyl 2-(bromomethyl)benzoate (1.24 g, 5.4 mmol) and K₂CO₃ (1.66 g, 12 mmol) in DMF (10 ml) was stirred at room temperature for 3 hours. The reaction mixture was diluted with water and extracted with ethyl acetate (×2). The combined organic layer was washed with brine, dried over Na₂SO₄, concentrated in vacuo. The crude product (1.6 g, 100%) was used in the next reaction without further purification.

### Intermediate 5

¹H NMR (600 MHz, CHLOROFORM-d) δ ppm 3.87 (s, 3H), 4.24 (s, 2H), 4.45 (br. s., 2H), 6.24 (td, J=8.4, 1.6 Hz, 1H), 6.36 (dd, J=10.4, 1.6 Hz, 1H), 6.94 (d, J=7.3 Hz, 1H), 7.00 (t, J=7.5 Hz, 1H), 7.25-7.34 (m, 2H), 7.89 (d, J=7.6 Hz, 1H).

### Example 17

### Compound 13

### methyl 2-[({2-[(1-benzothiophen-2-ylsulfonyl)amino]-4-fluorophenyl}sulfanyl)methyl]benzoate

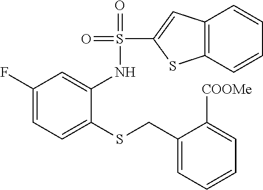 [SMILES: COC(=O)c1ccccc1CSc1ccc(F)cc1NS(=O)(=O)c1cc2ccccc2s1]

A mixture of methyl 2-(((2-amino-4-fluorophenyl)thio)methyl)benzoate (Intermediate 5, 350 mg, 1.20 mmol) and benzo[b]thiophene-2-sulfonyl chloride (280 mg, 1.20 mmol) in pyridine (2.5 ml) was stirred at room temperature for 16 hours and was concentrated. The residue was diluted in EtOAc, washed successively with 1M HCl and brine, dried over Na₂SO₄, and concentrated. The crude product was purified by column chromatography on silica gel (0>20% EtOAc-hexane) to yield the title compound (364 mg, 62%).

¹H NMR (600 MHz, CHLOROFORM-d) δ ppm 3.93 (s, 3H), 4.14 (s, 2H), 6.63 (td, J=8.2, 2.6 Hz, 1H), 6.73-6.79 (m, 1H), 7.15 (dd, J=8.5, 6.2 Hz, 1H), 7.26-7.32 (m, 2H), 7.38-7.46 (m, 2H), 7.48 (dd, J=10.6, 2.6 Hz, 1H), 7.77 (d, J=8.2 Hz, 1H), 7.83 (d, J=8.2 Hz, 1H), 7.89 (s, 1H), 7.92-7.99 (m, 1H), 8.43 (s, 1H).

### Example 18

### Compound 14

### 2-[({2-[(1-benzothiophen-2-ylsulfonyl)amino]-4-fluorophenyl}sulfanyl)methyl]benzoic acid

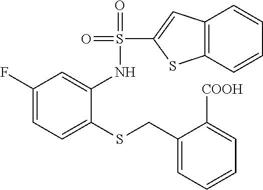 [SMILES: O=C(O)c1ccccc1CSc1ccc(F)cc1NS(=O)(=O)c1cc2ccccc2s1]

To methyl 2-[({2-[(1-benzothiophen-2-ylsulfonyl)amino]-4-fluorophenyl}sulfanyl)methyl]benzoate (Compound 13, 320 mg, 0.66 mmol) in MeOH (20 ml) was added 4M NaOH (4.1 ml) and the reaction was stirred at 45° C. for 3 hours. The reaction was cooled to room temperature, acidified with 6M HCl, and was concentrated. The residue was diluted with EtOAc, washed successively with water and brine, dried over Na₂SO₄, and concentrated. The crude product was purified by recrystallization from MeOH—CH₂Cl₂ to yield the title compound (290 mg, 93%).

¹H NMR (600 MHz, CHLOROFORM-d) δ ppm 4.18 (s, 2H), 6.65 (td, J=8.1, 2.1 Hz, 1H), 6.88 (d, J=6.5 Hz, 1H), 7.23 (dd, J=8.2, 6.7 Hz, 1H), 7.31-7.46 (m, 4H), 7.50 (dd, J=10.4, 1.9 Hz, 1H), 7.77 (d, J=8.2 Hz, 1H), 7.83 (d, J=7.6 Hz, 1H), 7.91 (s, 1H), 8.07-8.15 (m, 1H), 8.69 (s, 1H).

### Biological Examples

HEK-Gqi5 cells stably expressing CCR2 were cultured in DMEM high glucose, 10% FBS, 1% PSA, 400 μg/ml geneticin and 50 μg/ml hygromycin. Appropriate positive control chemokines (MCP-1, MIP1A or RANTES) was used as the positive control agonist for screening compound-induced calcium activity assayed on the FLIPR^{Tetra}. The drug plates were prepared in 384-well microplates using the EP3 and the MultiPROBE robotic liquid handling systems. Compounds were synthesized and tested for CCR2 activity.

TABLE 1
Biological activity of compounds (This
table is inclusive of old/new material)

| | CCR2 | CCR2 |
|---|---|---|
| | IC50 | ANTAGONISM |
| IUPAC Name | (nM) | Rel. Eff. (%) |
| methyl 2-[({2-[(1-benzothiophen-2- | 198 | 86 |
| ylsulfonyl)amino]-4-chloro- | | |
| phenyl}sulfanyl)methyl]benzoate | | |
| 2-[({2-[(1-benzothiophen-2- | 4 | 100 |
| ylsulfonyl)amino]-4-chloro- | | |
| phenyl}sulfanyl)methyl]benzoic acid | | |
| 2-[({2-[(1-benzothiophen-2- | nd | 99 |
| ylsulfonyl)amino]-4-methyl- | | |
| phenyl}sulfanyl)methyl]benzoic acid | | |
| N-{2-[(3-aminobenzyl)sulfonyl]-5- | 77 | 98 |
| chlorophenyl}-1-benzothiophene-2- | | |
| sulfonamide | | |
| 2-[({2-[(1-benzothiophen-2- | 114 | 88 |
| ylsulfonyl)amino]-5-methyl- | | |
| phenyl}sulfanyl)methyl]benzoic acid | | |
| N-[2-(benzylsulfanyl)pyridin-3-yl]-1- | 2254 | 44 |
| benzothiophene-2-sulfonamide | | |
| N-[2-(benzylsulfonyl)pyridin-3-yl]-1- | 85 | 93 |
| benzothiophene-2-sulfonamide | | |
| N-[2-(benzylsulfinyl)pyridin-3-yl]-1- | 122 | 72 |
| benzothiophene-2-sulfonamide | | |
| methyl 2-[({2-[(1-benzothiophen-2- | 268 | 68 |
| ylsulfonyl)amino]-4-fluoro- | | |
| phenyl}sulfanyl)methyl]benzoate | | |
| 2-[({2-[(1-benzothiophen-2- | <10 | 96 |
| ylsulfonyl)amino]-4-fluoro- | | |
| phenyl}sulfanyl)methyl]benzoic acid | | |

nd: not determinable.

What is claimed is:

1. A compound represented by Formula I, its individual enantiomers, individual diastereoisomers, individual tautomers or a pharmaceutically acceptable salt thereof: 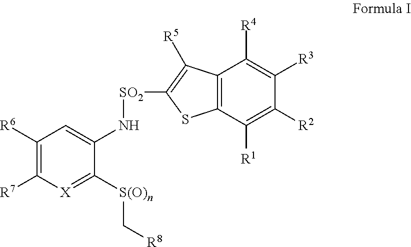
wherein:
X is N or CR;
R is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R¹ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R² is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R³ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁴ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁵ is hydrogen, halogen, CN, substituted or unsubstituted C₁₋₆ alkyl, substituted or unsubstituted Cₘ cycloalkyl, OR⁹, NR¹⁰R¹¹ or COR¹²;
R⁶ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁷ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁸ is substituted or unsubstituted C₁₋₆ alkyl, substituted or unsubstituted C₃₋₈ cycloalkyl, substituted or unsubstituted aryl or substituted or unsubstituted heteroaryl;
n is 0, 1 or 2;
R⁹ is hydrogen or substituted or unsubstituted C₁₋₆ alkyl;
R¹⁰ is hydrogen or substituted or unsubstituted C₁₋₆ alkyl;
R¹¹ is hydrogen, substituted or unsubstituted C₁₋₆ alkyl, substituted or unsubstituted heterocycle or substituted or unsubstituted C₆₋₁₀ aryl;
R¹² is hydrogen, hydroxyl, substituted or unsubstituted heterocycle, substituted or unsubstituted C₆₋₁₀ aryl or substituted or unsubstituted C₁₋₆ alkyl;
except compounds:

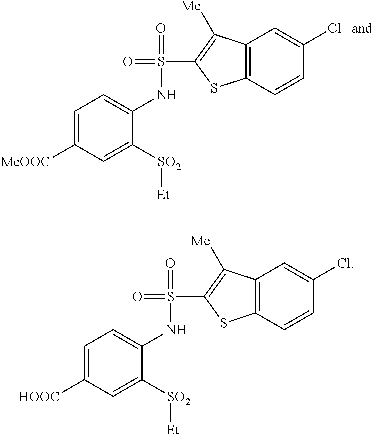 [SMILES: CCS(=O)(=O)c1cc(C(=O)OC)ccc1NS(=O)(=O)c1sc2ccc(Cl)cc2c1C ; CCS(=O)(=O)c1cc(C(=O)O)ccc1NS(=O)(=O)c1sc2ccc(Cl)cc2c1C]

2. A compound according to claim 1 wherein:
n is 0.

3. A compound according to claim 1 wherein:
X is CR; and
R is hydrogen.

4. A compound according to claim 1 wherein:
X is CR;
R is hydrogen; and
n is 0.

5. A compound according to claim 1 selected from:
methyl 2-[({2-[(1-benzothiophen-2-ylsulfonyl)amino]-4-fluorophenyl}sulfanyl)methyl]benzoate;
2-[({2-[(1-benzothiophen-2-ylsulfonyl)amino]-4-fluorophenyl}sulfanyl)methyl]benzoic acid;
N-[2-(Benzylsulfinyl)pyridin-3-yl]-1-benzothiophene-2-sulfonamide;
N-[2-(Benzylsulfonyl)pyridin-3-yl]-1-benzothiophene-2-sulfonamide;
N-[2-(Benzylsulfanyl)pyridin-3-yl]-1-benzothiophene-2-sulfonamide;
2-[({2-[(1-Benzothiophen-2-ylsulfonyl)amino]-5-methylphenyl}sulfanyl)methyl]benzoic acid;
Methyl 2-[({2-[(1-Benzothiophen-2-ylsulfonyl)amino]-5-methylphenyl}sulfanyl) methyl]benzoate;
N-{2-[(3-Aminobenzyl)sulfonyl]-5-chlorophenyl}-1-benzothiophene-2-sulfonamide;
2-[({2-[(1-Benzothiophen-2-ylsulfonyl)amino]-4-methylphenyl}sulfanyl)methyl]benzoic acid;
N-(5-Chloro-2-((3-nitrobenzyl)thio)phenyl)benzo[b]thiophene-2-sulfonamide;
N-(5-Chloro-2-((3-nitrobenzyl)sulfonyl)phenyl)benzo[b]thiophene-2-sulfonamide;
Methyl 2-[({2-[(1-benzothiophen-2-ylsulfonyl)amino]-4-methylphenyl}sulfanyl) methyl]benzoate;
2-[({2-[(1-Benzothiophen-2-ylsulfonyl)amino]-4-chlorophenyl}sulfanyl)methyl]benzoic acid; and
Methyl 2-[({2-[(1-benzothiophen-2-ylsulfonyl)amino]-4-chlorophenyl}sulfanyl) methyl]benzoate.

6. A pharmaceutical composition comprising as active ingredient a therapeutically effective amount of a compound according to claim 1 and a pharmaceutically acceptable adjuvant, diluent or carrier.

7. A pharmaceutical composition according to claim 6 wherein the compound is selected from:
methyl 2-[({2-[(1-benzothiophen-2-ylsulfonyl)amino]-4-fluorophenyl}sulfanyl)methyl]benzoate;
2-[({2-[(1-benzothiophen-2-ylsulfonyl)amino]-4-fluorophenyl}sulfanyl)methyl]benzoic acid;
N-[2-(Benzylsulfinyl)pyridin-3-yl]-1-benzothiophene-2-sulfonamide;
N-[2-(Benzylsulfonyl)pyridin-3-yl]-1-benzothiophene-2-sulfonamide;
N-[2-(Benzylsulfanyl)pyridin-3-yl]-1-benzothiophene-2-sulfonamide;
2-[({2-[(1-Benzothiophen-2-ylsulfonyl)amino]-5-methylphenyl}sulfanyl)methyl]benzoic acid;
Methyl 2-[({2-[(1-Benzothiophen-2-ylsulfonyl)amino]-5-methylphenyl}sulfanyl) methyl]benzoate;
N-(5-Chloro-2-((3-nitrobenzyl)thio)phenyl)benzo[b]thiophene-2-sulfonamide;
N-(5-Chloro-2-((3-nitrobenzyl)sulfonyl)phenyl)benzo[b]thiophene-2-sulfonamide;
N-{2-[(3-Aminobenzyl)sulfonyl]-5-chlorophenyl}-1-benzothiophene-2-sulfonamide;
2-[({2-[(1-Benzothiophen-2-ylsulfonyl)amino]-4-methylphenyl}sulfanyl)methyl]benzoic acid;
Methyl 2-[({2-[(1-benzothiophen-2-ylsulfonyl)amino]-4-methylphenyl}sulfanyl) methyl]benzoate;
2-[({2-[(1-Benzothiophen-2-ylsulfonyl)amino]-4-chlorophenyl}sulfanyl)methyl]benzoic acid;
Methyl 2-[({2-[(1-benzothiophen-2-ylsulfonyl)amino]-4-chlorophenyl}sulfanyl) methyl]benzoate.

8. A method of treating a disorder associated with chemokine receptor modulation, which comprises administering to a mammal in need thereof, a pharmaceutical composition comprising a therapeutically effective amount of at least one compound of Formula I 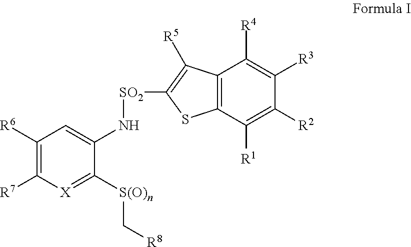
wherein:
X is N or CR;
R is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R¹ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R² is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R³ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁴ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁵ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁶ is hydrogen, halogen, CN, substituted or unsubstituted C₁₋₆ alkyl, substituted or unsubstituted C₃₋₈ cycloalkyl, OR⁹, NR¹⁰R¹¹ or COR¹²;
R⁷ is hydrogen, halogen, CN, substituted or unsubstituted C₁₋₆ alkyl, substituted or unsubstituted Cₘ cycloalkyl, OR⁹, NR¹⁰R¹¹ or COR¹²;
R⁸ is substituted or unsubstituted C₁₋₆ alkyl, substituted or unsubstituted C₃₋₈ cycloalkyl, substituted or unsubstituted aryl or substituted or unsubstituted heteroaryl;
n is 0, 1 or 2;
R⁹ is hydrogen or substituted or unsubstituted C₁₋₆ alkyl;
R¹⁰ is hydrogen or substituted or unsubstituted C₁₋₆ alkyl;
R¹¹ is hydrogen, substituted or unsubstituted C₁₋₆ alkyl, substituted or unsubstituted heterocycle or substituted or unsubstituted C₆₋₁₀ aryl;
R¹² is hydrogen, hydroxyl, substituted or unsubstituted heterocycle, substituted or unsubstituted C₆₋₁₀ aryl or substituted or unsubstituted C₁₋₆ alkyl; except compounds:

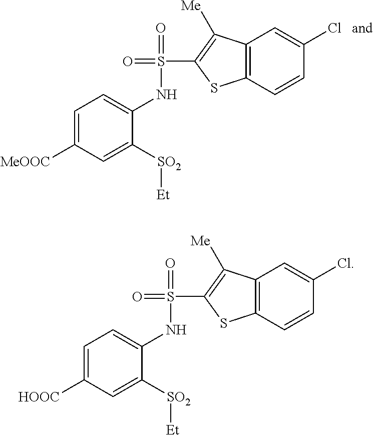 [SMILES: CCS(=O)(=O)c1cc(C(=O)OC)ccc1NS(=O)(=O)c1sc2ccc(Cl)cc2c1C ; CCS(=O)(=O)c1cc(C(=O)O)ccc1NS(=O)(=O)c1sc2ccc(Cl)cc2c1C]

9. A method of claim 8, wherein the pharmaceutical composition is administered to the mammal to treat ocular inflammatory diseases and skin inflammatory diseases and conditions.

10. The method of claim 8 wherein the mammal is a human.
